(19) United States
(12) Patent Application Publication
(10) Pub. No.: US 2026/0022083 A1
(43) Pub. Date: Jan. 22, 2026

(54) EMISSIONS-FREE OLEFIN SYNTHESIS VIA CONCENTRATED SOLAR-THERMAL PYROLYSIS

(71) Applicants: SolGrapH, Inc., Los Angeles, CA (US); The Regents of the University of California, Oakland, CA (US)

(72) Inventors: Anil Pradeep Nair, Oak Park, IL (US); Raymond Mitchell Spearrin, Calabasas, CA (US); Barathan Jeevaretanam, Kuala Lumpur (MY)

(21) Appl. No.: 19/271,986

(22) Filed: Jul. 17, 2025

Related U.S. Application Data
(60) Provisional application No. 63/672,939, filed on Jul. 18, 2024.

(51) Int. Cl. C07C 5/327

(52) U.S. Cl. CPC C07C 5/327

(57) ABSTRACT

The present disclosure relates to a novel technology that uses concentrated solar radiation to convert hydrocarbons into olefins (such as ethylene and propylene), which are precursors to a myriad of industrial products, including plastics and resins. This process is a disruptive, emissions-free alternative to conventional steam-cracking olefin production that emits high levels of CO₂, instead generating a valuable solid carbon co-product that can be used for battery electrodes and other applications.

### PRIORITY CLAIM

The present application claims the benefit of U.S. Provisional Application No. 63/672,939 filed Jul. 18, 2024, which is hereby incorporated by reference herein in its entirety.

### TECHNICAL FIELD

The present disclosure is generally directed to production of olefins.

### BACKGROUND

Light olefins, namely ethylene and propylene, are the most common organic chemicals used in the chemical industry. Ethylene and propylene are direct precursors to high/low-density polyethylene (HDPE/LDPE) and polypropylene (PP) respectively, which combined represent around 50% of global plastic production. Among several other industrial uses, the molecules are also chemically converted into other plastic monomers, such as ethylene glycol for polyethylene terephthalate (PET), ethylene dichloride for polyvinyl chloride (PVC), and styrene for polystyrene (PS), representing another 25% of global plastics production. The demand for olefins, particularly propylene, is projected to increase in the coming decades as the global population and standards of living continue to rise.

Nearly all ethylene in the US is produced via steam cracking of natural gas liquid (NGL) components such as ethane and propane, with propylene produced as a byproduct. In steam cracking, the highly saturated NGL hydrocarbons are heated to high temperatures that promote the dehydrogenation of alkanes into olefins. Increased heat transfer resistance can occur due to inadvertent amorphous carbon deposition (coke) on the hot furnace walls, which can impede the dehydrogenation reaction. To mitigate this problem, high-temperature steam is co-flowed with the hydrocarbons. The steam reacts with the coke to form carbon oxides, which result in direct CO₂ emissions. The steam also must be generated and heated along with the hydrocarbon feedstock, resulting in a higher process heat requirement. As such, 1-2 kg of CO₂ is released per kg of ethylene produced, amounting to over 260 Mt of annual CO₂ emissions, or nearly 1% of global greenhouse gas (GHG) emissions.

With the growing global and domestic demand for plastic products and the growing US supply of oil and gas, the CO₂ emissions impact of domestic olefin production is anticipated to increase unless new clean olefin production technologies are deployed. In particular, PP demand is projected to grow faster than HDPE/LDPE demand in the coming years, due in part to PP's excellent strength-to-weight and durability making it useful for automotive, consumer electronic, and textile applications. Unfortunately, due to the difficulty in recovering PP at the end-of-life of these products, there is limited opportunity to meet increased PP demand via recycling, highlighting the acute need for cleaner synthesis of virgin polypropylene feedstock.

### SUMMARY

The present disclosure relates to a novel technology that uses concentrated solar radiation to convert hydrocarbons into olefins (such as ethylene and propylene), which are precursors to a myriad of industrial products, including plastics and resins. This process is a disruptive, emissions-free alternative to conventional steam-cracking olefin production that emits high levels of CO₂, instead generating a valuable solid carbon co-product that can be used for battery electrodes and other applications.

Disclosed herein is a novel solar-thermal reforming process via direct irradiation of a porous light absorber, which also serves as the gas reaction site. Hydrocarbon gas is flowed through the heated porous absorber and subsequently pyrolyzed due to the localized high temperatures. The use of a thin porous absorber as the reaction site exhibits the following advantages: 1) a high specific-area heat transfer medium between the incident solar irradiation and flowing gas, which enables high rates of gas decomposition and solid-product deposition per unit volume; 2) an extractable site for solid carbon deposition, facilitating solid-product extraction and mitigating unwanted deposition on other reactor surfaces; 3) a thin cross-sectional area that limits conduction losses to the surrounding reactor chamber walls; and 4) low thermal mass that facilitates rapid reaction startup upon incident solar irradiation. Moreover, due to the limited residence time (tens of milliseconds) of the feedstock in the thin reaction zone, the decomposition of the feedstock in this process can be kinetically-limited. This gives rise to non-equilibrium gas mixtures in the product stream, resulting in product streams that contain a mixture of species reflecting variable reaction progress. Product yields of target species can be tuned by variations in feedstocks and process parameters such as feedstock flow rate, incident heat flux, and chamber pressure.

In an embodiment, a method of producing olefins can include generating ethylene from ethane. The ethylene can be collected along with co-products including graphitic carbon and hydrogen.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 depicts a solar-thermal olefin production process flow according to the disclosure, including a depiction of a 50-kW Southwest Solar Technology solar dish concentrator that can be used in the process in the top left corner.

FIG. 2a depicts a schematic of a laboratory roll-to-roll solar-thermal olefin syntheses experimental setup according to the disclosure.

FIG. 2b depicts a photograph of a High Flux Solar Simulator (top) and a solar-thermal reactor (bottom) according to the disclosure.

FIG. 3 depicts hydrocarbon and hydrogen exhaust mass fractions in an ethane to ethylene conversion according to the disclosure.

While various embodiments are amenable to various modifications and alternative forms, specifics thereof have been shown by way of example in the drawings and will be described in detail. It should be understood, however, that the intention is not to limit the disclosed inventions to the particular embodiments described. On the contrary, the intention is to cover all modifications, equivalents, and alternatives falling within the spirit and scope of the subject matter described herein.

### DETAILED DESCRIPTION

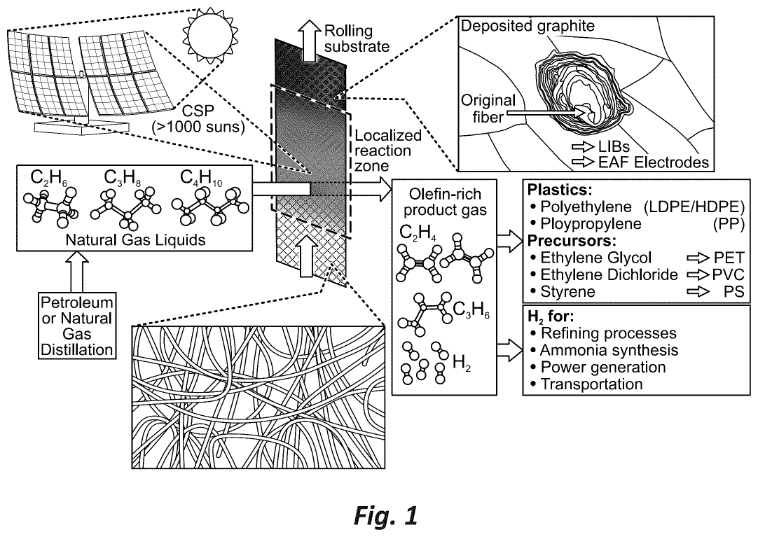

Disclosed herein is a solar-thermal olefin production process by which direct concentrated solar irradiation is used to convert a hydrocarbon gas mixture into light olefins with zero CO₂ emissions. This solar-thermal process contrasts conventional olefin production methods including steam cracking, fluid catalytic cracking, and propane dehydrogenation, which all involve direct CO₂ emissions. Referring to FIG. 1, the solar-thermal process involves irradiating a thin, porous substrate (for example, a carbon cloth/felt) with concentrated solar light to generate high temperatures. In embodiments, the hydrocarbon gas mixture can be one or more saturated hydrocarbons, such the ethane, propane, and butane which make up the major constituents of natural gas liquid (NGL). These hydrocarbons can be obtained through petroleum or natural gas distillation. The hydrocarbon gas mixture flows through the high specific area porous medium, inducing a rapid pyrolysis reaction that dehydrogenates the alkanes in the hydrocarbon gas into olefins without the use of a catalyst. The resulting olefin-rich product gas can be used for the production of plastics or other industrial processes. In some embodiments, non-hydrocarbon gases can be included in the gas mixture. These additional gases may be present as impurities in the feedstock or may be intentionally blended into the feedstock to tune product yields and/or to mitigate the production of unwanted co-products.

Of the olefins, ethylene (C₂H₄) has the largest global demand as it is the monomer of the commonly used plastic polyethylene (PE). It is also precursor to ethylene glycol (a precursor for the plastic polyethylene terephthalate (PET)), ethylene dichloride (a precursor for the plastic polyvinyl chloride (PVC)) and styrene (the monomer of the plastic polystyrene (PS)). Ethylene is chiefly produced via steam cracking of ethane or naphtha where the feedstock is heated in a tubular reactor to 1000-1200K, which cracks and dehydrogenates the hydrocarbons into ethylene. Typically, heat is provided for the process through the external walls of the steam cracker reactor. A chief concern with the process is the generation of amorphous carbon in the form of coke on the walls of the steam cracker reactor, which is at higher temperatures than the bulk reactant gas. This coke buildup can cause a degradation of reactor performance over time due to reduction in heat transfer capability. As such, coke buildup must be periodically removed to ensure reactor operability. To suppress coke formation, the steam cracking process involves the co-flow of steam at 20-30% weight fraction with the hydrocarbon mixture. The high-temperature steam oxidizes the coke into CO and CO₂ to slow the net rate of coke deposition. Nevertheless, modern steam crackers still need to be de-coked several times a year, which can result in weeks of operational downtime. The addition of steam leads to significant CO₂ emissions, both via direct carbon oxidation and by the increased process heat requirement to boil and superheat the steam, which is typically supplied by burning some of the steam-cracker product gas.

The solar-thermal process described herein contrasts with steam cracking, in that the highly localized solar heating of the substrate prevents pyrolysis reactions from occurring on the reactor walls, thus mitigating coke buildup and the need for periodic de-coking. As such, no steam co-flow is required, allowing for more thermal energy to be used directly for hydrocarbon cracking instead of steam generation and heating, resulting in a more thermally efficient process. The lack of steam in the reactor also eliminates direct CO₂ emissions by preventing the reaction of solid carbon with the oxygen contained in steam. Because the solar-thermal process requires zero direct or indirect CO₂ emissions, the process is carbon neutral. With a biomass-derived feedstock, the process could be carbon-negative to aid in global and domestic decarbonization goals. While several alterations to the steam cracking process have been proposed to abate some CO₂ emissions, such as furnace heat generation via renewable electricity, these methods do not eliminate the direct CO₂ emissions associated with process carbon oxidation and suffer from rising marginal electricity prices.

As the monomer for polypropylene (PP), propylene has the second highest global demand in terms of olefins. At present, propylene is produced using a broad mix of processes. Propylene can be produced as a byproduct of ethylene production in steam cracking but is also be produced as a byproduct in Fluid Catalytic Cracking (FCC) of heavy hydrocarbons or “on-purpose” via Propane Dehydrogenation (PDH). Both FCC and PDH involve the use of catalysts that reduce the required reaction temperature. This inherently reduces the amount of coke formation on reactor walls compared to steam cracking. However, the catalysts are “poisoned” over time due to coke deposition on the catalyst surface. As such, the catalyst must be periodically regenerated by burning off the coke with oxygen, producing direct CO₂ emissions. As the solar-thermal olefin production method disclosed herein is autocatalytic, it does not require catalyst regeneration, eliminating direct CO₂ emissions in the context of propylene production. The solar-thermal process can be tuned to produce larger quantities of propylene or other heavier olefins by including heavier hydrocarbons such as propane in the feedstock. Hydrocarbons that are liquid at standard temperature and pressure conditions may be vaporized into the gas phase prior to introduction into the solar-thermal reactor by increasing fluid temperature and reducing fluid pressure.

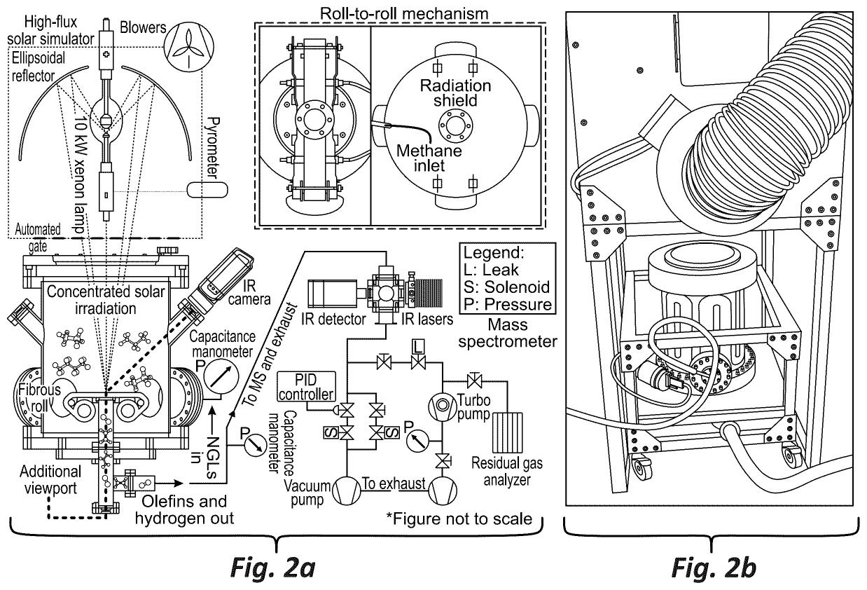

Incidental solid carbon generation does still occur on the porous substrate, but in contrast to steam cracking, this incidental carbon generation is a major benefit, not a liability: under certain conditions, carbon is deposited onto the substrate in a highly crystalline graphitic form (in contrast to amorphous coke), as confirmed via X-ray diffraction and Raman spectroscopy, and is suitable for several end applications such as lithium-ion battery anodes and electric-arc furnace electrodes. Scanning-electron microscopy images of the carbon substrate and deposited graphite are depicted in FIG. 1. To prevent substrate saturation with solid carbon product, the substrate is continuously replenished in the localized reaction zone via a roll-to-roll system, which is schematically depicted in FIG. 2a.

Although a secondary product, this graphitic solid carbon co-product can be used as a domestic source of clean electrode material for batteries and steel furnaces. The demand for graphite is expected to grow 5-10× over the next decade, driven by demand for electric vehicles and energy-storage solutions. Over 80% of today's natural graphite supply is sourced from China and is highly polluting (creating 5-10 kg CO₂ per kg of graphite produced). While synthetic graphite is a viable substitute for natural graphite, synthetic graphite today costs approximately 50% more than natural graphite at high volumes, and current production requires needle petroleum coke, which is only produced at a handful of refineries globally. Due to these supply risks, graphite has been included in the U.S. Department of Energy's list of critical materials/minerals for energy in the short and medium term. The proposed solar synthesis technology disclosed herein offers the opportunity to create entirely new domestic sources of clean and cost-effective plastic precursors and synthetic graphite to support the clean-energy transition using the large swathe of heavily insolated land area in the U.S. Southwest.

In addition to olefins and solid carbon, hydrogen is also produced as a co-product of the solar-thermal process, which can be used for other refinery processes such as hydrotreating or hydrocracking, or sold at a relatively low cost for transportation, ammonia synthesis, power generation, or heating. The clean hydrogen derived from this process can help meet growing demand for alternatives to steam-methane reforming (SMR), which involves substantial CO₂ emissions, and in doing so will improve the economics of solar-thermal olefin synthesis.

In some embodiments additional gases other than hydrogen and additional solids other than carbon can be generated as co-products. This may occur with the use of non-carbonaceous substrate materials or with the addition of non-hydrocarbon gases into the feedstock.

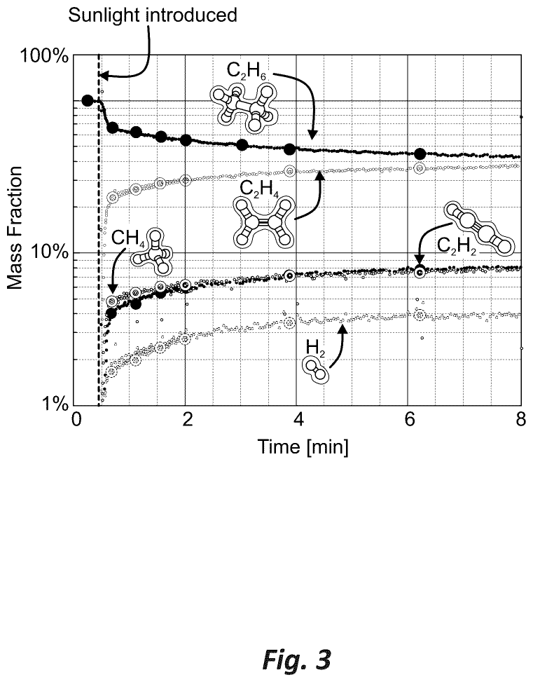

The inventors have previously demonstrated autocatalytic solar-thermal pyrolysis in the context of natural gas decomposition to produce graphite and hydrogen at higher temperatures using UCLA's High-Flux Solar Simulator (HFSS) facility. Recently, olefin production has been demonstrated using the HFSS with an ethane feedstock. The setup for this demonstration is schematically depicted in FIGS. 2a-2b. This figure depicts the HFSS providing a surrogate source of concentrated solar irradiation to a fibrous substrate web which is translated through the reaction zone by a roll-to-roll mechanism. In this example, the hydrocarbon feedstock is comprised solely of ethane. The ethane flows through the fibrous web with hydrogen and the olefin ethylene exhausted out. Graphitic carbon is deposited onto the fibrous web. A fluid control system involving mass flow controllers, pressure transducers, and vacuum pumps is used to control process parameters such as feedstock mass flow rate and reaction chamber pressure, which modulate synthesis conditions. Some of these fluid components are controlled with a PID controller to ensure that the process parameters are maintained at the desired setpoint during operation. Reaction temperature can be controlled by varying the solar simulator power input, which modulates the intensity of the light incident on the porous substrate, which is analogous to defocusing the concentrated solar radiation in a real solar dish concentrator employed with natural sunlight. Diagnostics such as an infrared (IR) thermal camera, a mass spectrometer (MS), and infrared laser absorption spectroscopy (LAS) sensors were employed to monitor reaction temperature and products during operation. The LAS system was used to monitor the exhaust mole fractions of ethane, ethylene, acetylene, and methane. A molar balance of carbon and hydrogen atoms was used to infer the rates of hydrogen and solid carbon production. The results of one such experiment are shown in FIG. 3 in which ethylene is produced at a weight fraction of nearly 40%, which is comparable to yields from conventional steam cracking. While the preliminary results in FIG. 3 are promising, the process can be optimized in other embodiments with various other alternatives including: a) variations of synthesis parameters such as operating pressure, reactant flow rate, solar power, feedstock gas composition, and substrate material/thickness; b) the use of heavier hydrocarbons in the feedstock such as propane and butane to generate heavier olefins such as propylene and butylene; c) a post-reaction quench header to halt ethylene dehydrogenation to acetylene; d) optimization of process yields by equalizing of solar flux across the deposition substrate for temperature uniformity; and e) and the use of natural sunlight with a solar dish concentrator system.

In embodiments, a method of producing olefins can include irradiating a porous substrate to create a heated reaction zone. A hydrocarbon gas mixture can be caused to flow through the heated reaction zone in the substrate to induce a rapid pyrolysis reaction that dehydrogenates hydrocarbons in the hydrocarbon gas mixture into olefins without the use of a catalyst. The olefins can be collected along with co-products including carbon and hydrogen.

In some embodiments, the hydrocarbon gas mixture includes ethane.

In some embodiments, the hydrocarbon gas mixture includes propane.

In some embodiments, the hydrocarbon gas mixture is obtained by vaporization of a hydrocarbon liquid.

In some embodiments, the hydrocarbon gas mixture is diluted with non-hydrocarbon gases.

In some embodiments, the olefins include ethylene.

In some embodiments, the olefins include propylene.

In some embodiments, the co-products include other gases in addition to hydrogen.

In some embodiments, the solid carbon co-product is graphitic.

In some embodiments, the co-products include other solids in addition to carbon.

In some embodiments, a flow rate of the hydrocarbon gas mixture can be controlled.

In some embodiments, the flow rate of the hydrocarbon gas mixture is controlled in order to control an amount of time that the hydrocarbon gas mixture is within the heated reaction zone.

In some embodiments, a constant target pressure within the heated reaction zone can be maintained while the hydrocarbon gas mixture flows through the heated reaction zone.

In some embodiments, a heat flux within the heated reaction zone can be controlled while the hydrocarbon gas mixture through the heated reaction zone.

In some embodiments, controlling the heat flux within the heated reaction zone provides indirect control of a temperature in the heated reaction zone.

In some embodiments, the rapid pyrolysis reaction can be halted when olefins have formed.

In some embodiments, the porous substrate is formed from carbon fibers.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

1. A method of producing olefins, comprising:
irradiating a porous substrate to create a heated reaction zone;
causing a hydrocarbon gas mixture to flow through the heated reaction zone in the substrate to induce a rapid pyrolysis reaction that dehydrogenates hydrocarbons in the hydrocarbon gas mixture into olefins without the use of a catalyst; and
collecting the olefins along with co-products including carbon and hydrogen.

2. The method of claim 1, wherein the hydrocarbon gas mixture includes ethane.

3. The method of claim 1, wherein the hydrocarbon gas mixture includes propane.

4. The method of claim 1, wherein the hydrocarbon gas mixture is produced by vaporization of a hydrocarbon liquid.

5. The method of claim 1, wherein the hydrocarbon gas mixture is diluted with non-hydrocarbon gases.

6. The method of claim 1, wherein the olefins include ethylene.

7. The method of claim 1, wherein the olefins include propylene.

8. The method of claim 1, wherein the co-products include other gases in addition to hydrogen.

9. The method of claim 1, wherein the solid carbon co-product is graphitic.

10. The method of claim 1, wherein the co-products include other solids in addition to carbon.

11. The method of claim 1, further comprising controlling a flow rate of the hydrocarbon gas mixture.

12. The method of claim 11, wherein the flow rate of the hydrocarbon gas mixture is controlled in order to control an amount of time that the hydrocarbon gas mixture is within the heated reaction zone.

13. The method of claim 1, further comprising maintaining a constant target pressure within the heated reaction zone while the hydrocarbon gas mixture flows through the heated reaction zone.

14. The method of claim 1, further comprising controlling a heat flux within the heated reaction zone while the hydrocarbon gas mixture through the heated reaction zone.

15. The method of claim 14, wherein controlling the heat flux within the heated reaction zone provides indirect control of a temperature in the heated reaction zone.

16. The method of claim 1, further comprising halting the rapid pyrolysis reaction when olefins have formed by quenching the gas.

17. The method of claim 1, wherein the porous substrate is formed from carbon fibers.
